(19) United States
(12) Patent Application Publication
(10) Pub. No.: US 2004/0185056 A1
(43) Pub. Date: Sep. 23, 2004

(54) Vaccines containing bovine herpesvirus 1 attenuated by mutation in latency-related gene

(75) Inventor: Clinton J. Jones, Lincoln, NE (US)

Correspondence Address: STINSON MORRISON HECKER LLP, ATTN: PATENT GROUP, 1201 WALNUT STREET, SUITE 2800, KANSAS CITY, MO, 64106-2150, US

(73) Assignee: Board of Regents, University of Nebraska-Lincoln

(21) Appl. No.: 10/752,482

(22) Filed: Jan. 5, 2004

Related U.S. Application Data
(60) Provisional application No. 60/437,855, filed on Jan. 3, 2003.

(51) Int. Cl. A61K 39/00; A61K 39/38

(52) U.S. Cl. 424/184.1

(57) ABSTRACT

Vaccines for pathogenic strains of bovine herpesvirus 1 (BHV-1) which are based on attenuated BHV-1 having a mutation in the latency-related gene are provided. Live, attenuated vaccines are also provided which express antigens from other viral or bacterial pathogens and thus form the basis of a variety of vaccines.

### CROSS-REFERENCE TO RELATED APPLICATIONS

This application is based on U.S. Provisional Application Serial No. 60/437,855, filed on Jan. 3, 2003 which is hereby incorporated herein by reference.

### STATEMENT REGARDING FEDERALLY SPONSORED RESEARCH OR DEVELOPMENT

This invention was made with government support under grants from the U.S. Department of Agriculture, numbers 9802064 and 2000-02060 and grant number P20RR15635 from the National Institutes of Health. The government has certain rights in this invention.

### BACKGROUND OF THE INVENTION

The present invention relates generally to vaccines based on live, attenuated viruses and to methods of using these vaccines. More particularly, the present invention relates to vaccines based on bovine herpesvirus 1, attenuated by mutation in the latency-related gene, and to methods for using these vaccines to induce immunological response to BHV-1 specific and/or recombinant immunogenic antigens without reactivation of the virus from latency.

The publications and other materials used herein to illuminate the background of the invention or provide additional details respecting the practice, are incorporated by reference, and for convenience are respectively grouped in the appended List of References.

Bovine herpesvirus 1 (BHV-1) is an important viral pathogen of cattle that can cause severe respiratory infection, conjunctivitis, abortions, vulvovaginitis, balanoposthitis, and systemic infection in neonate calves (Wyler et al., 1989). BHV-1 infection is also an important component of the upper respiratory tract infection referred to as “shipping fever” or bovine respiratory complex (Tikoo et al., 1995). BHV-1 is not the sole infectious agent associated with shipping fever, but it initiates the disorder by immuno-suppressing infected cattle, which results in secondary bacterial infections and pneumonia. Increased susceptibility to secondary infection correlates with depressed cell-mediated immunity after BHV-1 infection (Carter et al., 1989; Griebel et al., 1990; Griebel et al., 1987A; Griebel et al., 1987B). CD8⁺ T-cell recognition of infected cells is impaired by repressing expression of major histocompatibility complex class I and of the transporter associated with antigen presentation (Hariharan et al., 1993; Hinkley et al., 1998; Naturaj et al., 1997). CD4⁺ T-cell function is impaired during acute infection of calves, in part, because BHV-1 infects CD4⁺ T cells and induces apoptosis (Winkler et al., 1999). BHV-1 infection costs the cattle industry millions of dollars per year in the United States. Following acute infection, BHV-1 establishes lifelong latency in ganglionic neurons of the peripheral nervous system after initial replication in mucosal epithelium. Reactivation from latency results in virus shedding and transmission to other susceptible animals. Reactivation occurs after natural or corticosteroid-induced stress (Rock et al., 1992; Sheffy and Davies, 1972). Although modified live vaccines are available, there are disadvantages associated with the use of currently available vaccines, for example, they can cause disease in young calves or abortions in cows, and all have the potential to establish latency and reactivate from latency (Jones et al., 2000).

In order to overcome these disadvantages, improved live attenuated vaccines that are effective against BHV-1 and other viral and bacterial pathogens, and which will not reactivate from latency, are needed.

### BRIEF SUMMARY OF THE INVENTION

It is an object of the present invention to provide live viral vaccines based on BHV-1 attenuated by mutation in the latency-related (LR) gene so that the BHV-1 does not reactivate from latency. These live, attenuated viral vaccines cause acute infection and produce sufficient viral titer to elicit a cell-mediated immune response.

It is another object of the present invention to provide live viral vaccines based on BHV-1 attenuated by mutation in the LR gene which express recombinant immunogenic antigens. These attenuated viral vaccines cause acute infection and produce a specific antibody response to the recombinant antigens. The recombinant antigens may be one or more BHV-1 antigens or antigens of other viral or bacterial pathogens, or both.

It is a further object of the present invention to provide methods for effecting prophylactic treatment of viral or bacterial infection through the use of the live, attenuated viral vaccines of the present invention, wherein the vaccine is administered to an animal in need of treatment and induces immunological response to viral or bacterial pathogens.

According to the present invention, the foregoing and other objects are achieved by the discovery, as described in further detail herein, that the LR gene of BHV-1 is necessary for reactivation of the virus from latency, and that the LR mutant of BHV-1 is able to grow and cause acute intranasal infection, and is therefore able to elicit an immunological response in an infected animal. The vaccines of the present invention utilize live BHV-1, attenuated by mutation of the LR gene, to express BHV-1 specific and/or recombinant immunogenic antigens and, thus, induce immunological response to viral and bacterial pathogens. The vaccines of the present invention may be administered using one or more routes, including, but not limited to, subcutaneous, intramuscular, intranasal, intraperitoneal, and ocular.

### BRIEF DESCRIPTION OF THE DRAWINGS

FIG. 1 shows isolation and titration of virus present in trigeminal ganglia (TG) during acute infection. Virus was isolated from TG of mock, LR mutant, or wild-type (WT) infected calves on the designated days postinfection, as further described in Example 1. The titer of infectious virus was determined on bovine kidney cells (MDBK) as described in Example 1. The data shown are the averages from each group. A value of 0.2 was used to denote a negative result in order to visualize the bars.

FIG. 2 shows DNA sequences of the wild-type (wt) (SEQ ID NO:7) and LR mutant (SEQ ID NO:8). DNA was prepared from TG as described in Example 1. Primers P4 and P5 (Inman et al., 2001B) were used to amplify a region that contains the mutated sequences, including a unique EcoRI site. The amplified products were then digested with EcoRI (position in LR mutant is underlined) and visualized by ethidium bromide staining on 2% agarose gel electrophoresis.

FIG. 3 shows BHV-1 neutralizing antibody titers post infection and after DEX treatment, as indicated, measured as described in Example 1. Solid rectangles represent calves infected with wt virus; solid triangles represent LR-rescued virus; and open rectangles represent calves infected with the LR mutant virus. Each time point represents at least four calves. The inset panel is shown to illustrate the differences during acute phase more clearly (the x-axis scale is altered). The differences between the time points after 10 days postreactivation were statistically significant (P<0.05).

FIG. 4 shows viral shedding from ocular and nasal cavities after dexamethasone-induced reactivation determined using the procedures of Example 1. Shown are the percentages of calves that shed BHV-1 virus/total number of calves in each group on the designated day after dexamethasone (DEX) treatment. Four calves were used for the study with the LR-rescued virus, seven were used for the wt (WT) study, and seven were used for the study with the LR mutant.

### SUMMARY OF THE SEQUENCES

SEQ ID NO:1 is the nucleotide sequence for primer p4.

SEQ ID NO:2 is the nucleotide sequence for primer p5.

SEQ ID NO:3 is the nucleotide sequence for forward primer gC.

SEQ ID NO:4 is the nucleotide sequence for reverse primer gC.

SEQ ID NO:5 is the nucleotide sequence for forward primer gH.

SEQ ID NO:6 is the nucleotide sequence for reverse primer gH.

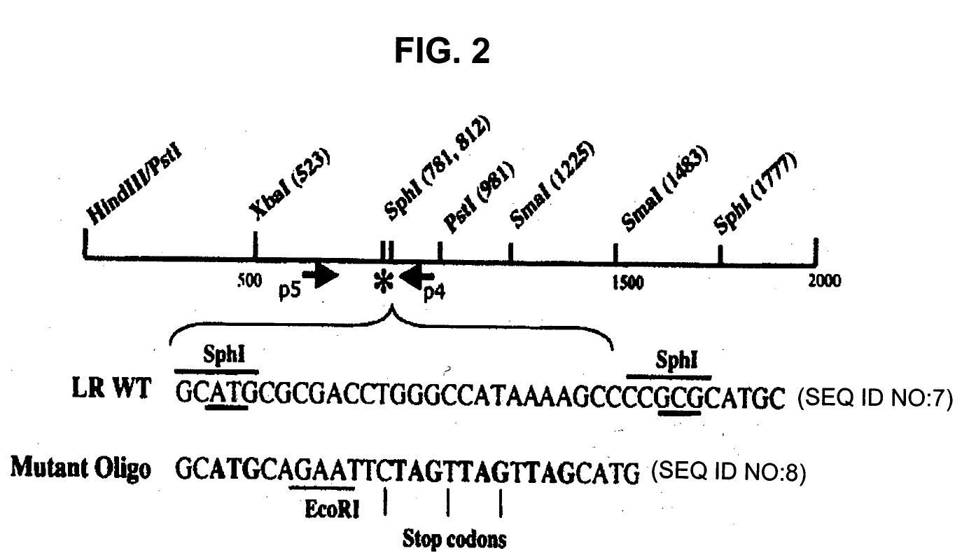

SEQ ID NO:7 is the nucleotide sequence of the wild-type BHV-1 latency-related ORF2 shown in FIG. 2.

SEQ ID NO:8 is the nucleotide sequence of the LR mutant oligonucleotide shown in FIG. 2.

### DETAILED DESCRIPTION OF PREFERRED EMBODIMENT

The present invention is directed to live, attenuated vaccines based on BHV-1 attenuated by mutation in the latency-related (LR) gene. The discovery, described in detail herein, that the LR gene of BHV-1 is not required to establish acute intranasal infection, and that the LR gene is essential for reactivation of BHV-1 from latency, makes possible the construction of live attenuated BHV-1 vaccines which are capable of inducing immune responses without reactivation from latency.

In one embodiment, the vaccines of the present invention provide immune system exposure to epitopes on the attenuated BHV-1 virus and induce immunological response to pathogenic strains of BHV-1. In another embodiment, the vaccines of the present invention provide immune system exposure to one or more recombinant antigens of BHV-1 and/or other pathogenic viruses or bacteria. The present invention can be practiced with a variety of viral and bacterial pathogens, including, without limitation, those which colonize and grow in the upper respiratory tract or other mucosal surfaces. Examples of such pathogens include bovine viral diarrhea virus (BVDV), bovine respiratory virus (BRSV), bovine corona viruses, and bacterial strains commonly associated with “Shipping Fever” (Pasteurella haemolytica, Pasteurella multocida, and Haemophilus somnus for example). The vaccines of the present invention are useful with mammals, preferably ungulates, most preferably with sheep, bovines, deer, and buffalo.

The present invention is further directed to methods for effecting treatment of infections through administration of the vaccines of the present invention to induce immunity in an animal in need of treatment. In one embodiment, the treatment is of a BHV-1 infection. In another embodiment, the treatment is of infection with a different pathogenic virus or a pathogenic bacterium.

BHV-1 is a member of the Alphaherpesvirinae subfamily and shares certain biological properties with herpes simplex virus types 1 and 2 (HSV-1 and -2, respectively) (Jones 1998). Viral gene expression in BHV-1 is temporally regulated in three distinct phases: immediate-early (IE), early (E), or late (L). Two IE transcription units exist: IE transcription unit 1 (IEtu1) and IEtu2. IEtu1 encodes functional homologues of two HSV-1 IE proteins, ICP0 and ICP4. IEtu2 encodes a protein that is similar to an essential HSV IE protein, ICP22 (Wirth et al., 1991). Bovine ICP0 (bICP0) is very important for productive infection, because it activates all classes of viral promoters and is expressed at high levels throughout infection (Fraefel et al., 1994; Wirth et al., 1992; Wirth et al., 1991).

Although the primary site of BHV-1 latency is sensory neurons, there is evidence that long-term persistence and reactivation also occur within germinal centers of pharyngeal tonsil (Winkler et al., 2000). The latency-related RNA (LR-RNA) is the only abundant viral transcript detected in latently infected neurons (Kutish et al., 1990; Rock et al., 1992; Rock et al., 1987A). A fraction of LR-RNA is polyadenylated and alternatively spliced in trigeminal ganglia (TG), suggesting this RNA is translated into more than one LR protein (LRP) (Devireddy and Jones, 1998; Hossain et al., 1995). LR gene products effect the normal cell cycle by inhibiting entry into S-phase. LRP is associated with cyclin-dependent kinase 2 (cdk2)-cyclin complexes (Hossain et al., 1995; Jiang et al., 1998). LR gene products also promote cell survival following induction of apoptosis in transiently transfected cells (Ciacci-Zanella et al, 1999).

Similar to BHV-1, HSV-1 establishes latency in ganglionic sensory neurons, typically TG or sacral dorsal root ganglia (Jones 1998; Wagner and Bloom, 1997). In situ hybridization has revealed that a small region, the latency-associated transcript (LAT), within the terminal repeats is abundantly transcribed in latently infected neurons (Rock et al., 1987B; Stevens et al., 1987). Numerous mutants that do not express detectable levels of LAT have been constructed. Although several studies have suggested that LAT plays no role in a latent infection, for example (Block et al., 1990; Ho and Mocarski, 1988), most have concluded that LAT is important, but not required. LAT enhances establishment of latency in mice (Sawtell, 1992; Thompson and Sawtell, 1997) and rabbits (Perng et al., 2000B), because certain LAT-null mutants contain lower levels of viral DNA in TG relative to wt virus (Devi-Rao et al., 1994; Maggioncalda et al., 1996). LAT interferes with apoptosis in transiently transfected cells and in infected mice or rabbits (Ahmed et al., 2002; Inman et al., 2001A; Perng et al., 2000A). The ability of LAT to interfere with apoptosis correlates with its ability to promote spontaneous reactivation (Inman et al., 2001A), suggesting the antiapoptotic activity of LAT has biological significance with respect to latency.

Although the LR gene of BHV-1 is homologue of the HSV-1 LAT gene, there are several notable differences. Most notably, the evidence presented herein shows that LR mutant BHV-1 does not reactivate from latency. In contrast, LAT-null mutants have been reported to reactivate from latency. Furthermore, the primary LAT transcript is 6.3 kb and includes two genes (Zwaagstra et al., 1990), whereas the primary LR transcript is only 1.5 kb and one gene (Kutish et al., 1990), and there is no obvious sequence homology between LR-RNA and LAT. While the region of LAT to which the spontaneous reactivation phenotype maps does not appear to encode a protein involved in the latency-reactivation cycle (Dorlet et al., 1998), the BHV-1 LR gene does encode such a protein (Jiang et al., 1998). Although an open reading frame in the stable 2-kb LAT may encode a protein (Coffin et al., 1998; Thomas et al., 1999), this protein is unlikely to play an important role in reactivation since it is located outside the region of LAT to which the reactivation function maps in rabbits (Bloom et al., 1996; Perng et al., 2001) and mice. Expression of an LR protein appears to play a role in protecting cells from death (Ciacci-Zanella et al., 1999). Finally, the LR gene has also been shown to inhibit cell cycle progression (Schang et al., 1996) and in the context of the viral genome plays an important role in virus shedding in the eyes of infected calves (Inman et al., 2001). Thus, structurally, the LR and LAT genes and RNAs are very different.

Definitions.

The present invention employs the following definitions, which are, where appropriate, referenced to BHV-1.

An “antigen” refers to a molecule containing one or more epitopes that will stimulate a host's immune system to make a secretory, humoral and/or cellular antigen-specific response.

The specific antigen can be a protein, a polysaccharide, a lipopolysaccharide or a lipopeptide; or it can be a combination of any of these. Particularly, the specific antigen can include a native protein or protein fragment, or a synthetic protein or protein fragment or peptide; it can include glycoprotein, glycopeptide, lipoprotein, lipopeptide, nucleoprotein, nucleopeptide; it can include a peptide-peptide conjugate; or it can include a recombinant nucleic acid expression product. Examples of antigens include, without limitation, those that are capable of eliciting an immune response against viral or bacterial bovine herpes virus, bovine respiratory virus, bovine viral diarrhea virus, bovine corona virus, and bacterial strains commonly associated with “shipping fever”.

The term “effective amount of an antigen” may be an amount capable of eliciting a demonstrable humoral, secretory, and/or cell-mediated immune response. The appropriate amount of antigen to be used is dependent on the specific antigen and is well known in the art.

“Encode.” A polynucleotide is said to “encode” a polypeptide if, in its native state or when manipulated by methods well known to those skilled in the art, it can be transcribed and/or translated to produce the mRNA for the polypeptide and/or a fragment thereof. The anti-sense strand is the complement of such a nucleic acid, and the encoding sequence can be deduced therefrom.

“Immunization” refers to the process of inducing a continuing protective level of antibody and/or cellular immune response which is directed against an antigen to which the organism has been previously exposed.

An “immunological response” to a vaccine is the development in the host of a humoral, secretory, and/or cell-mediated immune response to the vaccine of interest. An immunological response is usually assayed with an immunoassay. A variety of immunoassay methods are well known in the art. See, e.g., Harlow and Lane, 1988, or Goding, 1986.

“Latency-reactivation cycle” refers to a process operationally divided into three major steps: establishment of latency, maintenance of latency, and reactivation. Establishment of latency includes entry of the viral genome into a sensory neuron and acute infection. In BHV-1, viral gene expression is then generally extinguished, with the exception of the latency-related transcript (LR). Maintenance of latency is a phase that lasts for the life of the host and can be operationally defined as a period when infectious virus is not detected by standard virus isolation procedures. In general, abundant expression of viral genes that are required for productive infection does not occur during maintenance of latency. LR is the only known viral transcript that is abundantly expressed during this stage of latency. “Reactivation” and “reactivation from latency” refer to the process of viral reactivation which and stimulation of viral gene expression which occurs after exogenous administration of corticosteroids or elevated levels of natural corticosteroids as a consequence of stress. The synthetic corticosteroid dexamethasone (DEX) efficiently reactivates BHV-1 in latently infected rabbits and calves (Rock et al., 1992; Sheffy and Davies, 1972). Immunosuppression may also stimulate viral gene expression to cause reactivation. Upon reactivation, abundant viral gene expression may be detected in sensory neurons, and infectious BHV-1 virus can be isolated from TG, eye swabs, and/or nasal swabs. During reactivation, virus is translocated back to the initial site of infection, from which it can spread to other susceptible hosts. The ability to reactivate from latency results in recurrent disease and virus transmission.

“Operably linked” refers to a juxtaposition of components wherein the components so described are in a relationship permitting them to function in their intended manner. For instance, a promoter is operably linked to a coding sequence if the promoter affects its transcription or expression.

“Recombinant nucleic acid” is a nucleic acid which is not naturally occurring, or which is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. A polypeptide produced as an expression product of an isolated and manipulated genetic sequence is an “isolated polypeptide”, as used herein, even if expressed in a homologous cell type. Synthetically made forms or molecules expressing by heterologous cells are inherently isolated molecules.

A “recombinant vector” of the present invention is a live BHV-1 virus attenuated by mutation in the latency-related gene and expresses recombinant immunogenic antigens of one or more pathogen.

“Treatment” refers to the administration to an individual of a composition which yields a immunological response, and includes prophylaxis and/or therapy.

“Vaccine composition” and “vaccine” refer to an agent used to stimulate the immune system of an individual so that current harm is alleviated, or protection against future harm is provided.

As described in further detail herein (Example 4), and in Perng et al. (2002), incorporated herein in its entirety by reference, the LR gene was inserted into the LAT locus to determine whether it could restore spontaneous reactivation to a LAT-null mutant. It has been discovered that the LR gene of BHV-1 is capable of restoring high levels of spontaneous reactivation from latency to a LAT-null mutant, adding support to the hypothesis that inhibition of apoptosis plays an important role in the latency reactivation cycle of HSV-1 and BHV-1.

An LR mutant was constructed by inserting three stop codons near the beginning of the LR-RNA, suggesting that expression of LR proteins would be altered. The LR mutant grew with wild-type (wt) efficiency in bovine kidney cells (MDBK). Calves infected with the LR mutant consistently exhibited diminished clinical symptoms and a dramatic decrease (3 to 4 logs) in ocular, but not nasal, viral shedding occurred during acute infection compared to the wt or the LR-rescued virus (Inman et al., 2001). Conversely, the LR mutant had similar growth properties in productively infected bovine kidney cells (MDBK) and the nasal cavity of calves during acute infection. These results suggested that LR gene products promote virus growth in certain cell types in the eye or optic nerve during acute infection of cattle.

The evidence presented herein compares the latency reactivation cycle in calves infected with the LR mutant and to those from calves infected with wt BHV-1 or the LR-rescued virus. This application reports the results of examining the effects of the LR mutant on virus production in TG and the latency reactivation cycle. Diminished levels of virus were detected in TG of calves acutely infected with the LR mutant when compared to those infected with wt or LR-rescued virus. Although we consistently detected LR-RNA in TG of calves infected with the LR mutant or the wt by PCR, we were unable to detect viral DNA in neurons of calves infected with the LR mutant by in situ hybridization. PCR analysis confirmed that calves infected with the LR mutant contained lower levels of viral DNA during latency compared to those in calves infected with wt BHV-1. The evidence reported herein further shows that the LR mutant virus was not reactivated from latently infected calves following treatment with dexamethasone (DEX). In contrast, calves infected with wt virus or the LR-rescued virus reactivated efficiently following the same DEX treatment. The results described herein demonstrate that wt expression of LR gene products plays a crucial role in the latency reactivation cycle of BHV-1 in cattle.

As described herein in further detail, the LR mutant did not reactivate from DEX-induced reactivation, whereas the wt or LR-rescued virus did. The LR mutant contains three stop codons near the beginning of the LR-RNA that are designed to prohibit protein expression from all three reading frames (Inman et al., 2001B). The LR mutant also lacks 25 bp from wt sequence to prevent reversion to the wt. A peptide antibody that is directed against amino acid sequences within the LR open reading frame (ORF2) recognizes a protein of approximately 40 kDa in cells transfected with a wt LR gene construct (Ciacci-Zanella et al, 1999; Hossain et al., 1995; Jiang et al., 1998), but not when transfected with a plasmid containing the mutation used to make the LR mutant virus (Ciacci-Zanella et al, 1999). While not wanting to be bound by any particular theory, it appears that any phenotypic difference between the LR mutant and wt or LR-rescued virus is attributable to lack of protein expression by the LR mutant. Because the LR-RNA is alternatively spliced (Devireddy and Jones, 1998), there is the possibility that more than one protein is expressed. However, it is possible that differences in LR-RNA expression between wt BHV-1 and the LR mutant were responsible for the attenuated phenotype. Regardless of whether an LR protein or changes in LR-RNA mediate the altered phenotype of the LR mutant, the results reported herein clearly demonstrated that the latency reactivation cycle was disrupted following infection of calves with the LR mutant.

The TG is divided into three sections (ophthalmic, maxillary, and mandibular), and each section innervates the eye, nose, or mouth, respectively. Following infection of calves with the LR mutant, a 3- to 4-log reduction in virus shedding from ocular swabs was detected compared to the level in calves infected with wt or LR-rescued virus (Inman et al., 2001B). In contrast, shedding of the LR mutant from the nasal cavity during acute infection was not significantly different from that of wt BHV-1 or the LR-rescued virus. Without being bound to a particular mode of action, the results reported herein suggest that “seeding” of the TG by the LR mutant would be fairly normal via the maxillary route following infection, but lower levels of virus would seed the TG via the ocular route. Considering that it was not possible to reactivate the LR mutant and lower levels of viral DNA were detected in TG of calves latently infected with the LR mutant, it may be that seeding of viral DNA via the ocular route is more important. It is also possible that regardless of whether the LR mutant seeds the TG via the maxillary or ocular route, efficient amplification of the viral genome or reactivation from latency would not occur. The finding that 10- to 100-fold less infectious virus was present in TG of calves acutely infected with the LR mutant supports the contention that wt expression of LR gene products was important for acute infection of sensory neurons.

Several reports have demonstrated that HSV-1 LAT plays an important role in establishing (versus reactivation) latency (Perng et al., 2000B; Sawtell, 1997; Sawtell, 1992), suggesting LR gene products also play a role in establishing latency. Two findings in this study support a role for wt expression of LR gene products in the establishment of latency. First, reduced levels of viral DNA were detected in calves latently infected with the LR mutant. Second, the failure to detect in situ hybridization-positive neurons in calves latently infected with the LR mutant suggested that LR gene products were necessary for latency in neurons containing high copies of viral DNA. While not wanting to be bound by a particular mode of action, it is possible that LR gene products promote (i) productive infection in certain neurons, (ii) survival of “permissive” neurons that support productive infection, or (iii) infection of certain types of neurons. Considering that LR gene products interfere with apoptosis in transiently transfected cells (Ciacci-Zanella et al, 1999) and HSV-1 LAT interferes with apoptosis (Ahmed et al., 2002; Inman et al., 2001A; Perng et al., 2000A), it is tempting to speculate that LR gene products promote neuronal survival in TG of infected calves during acute infection.

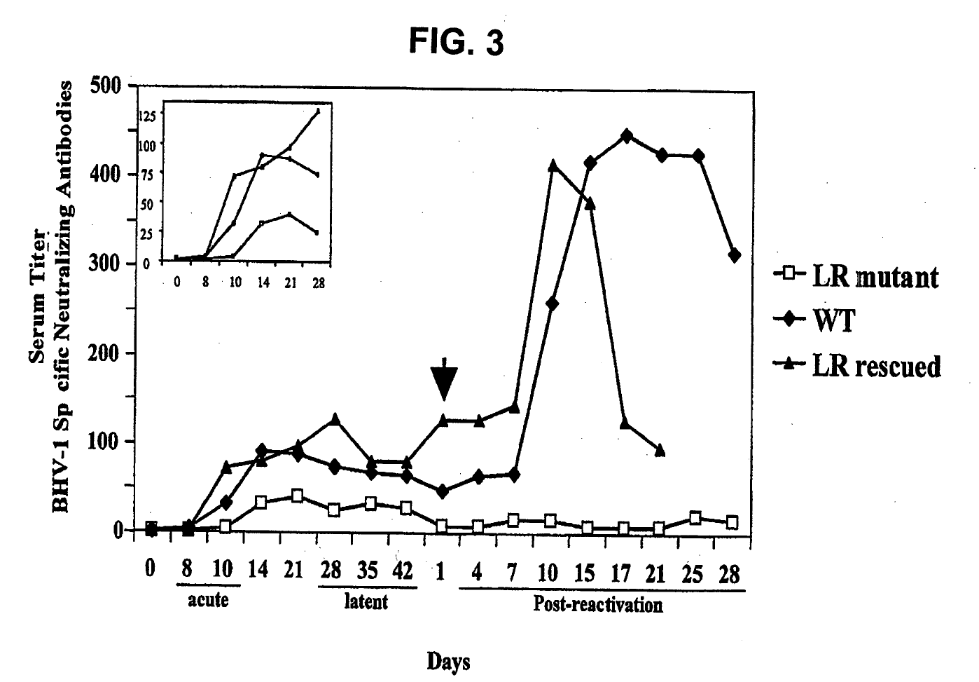

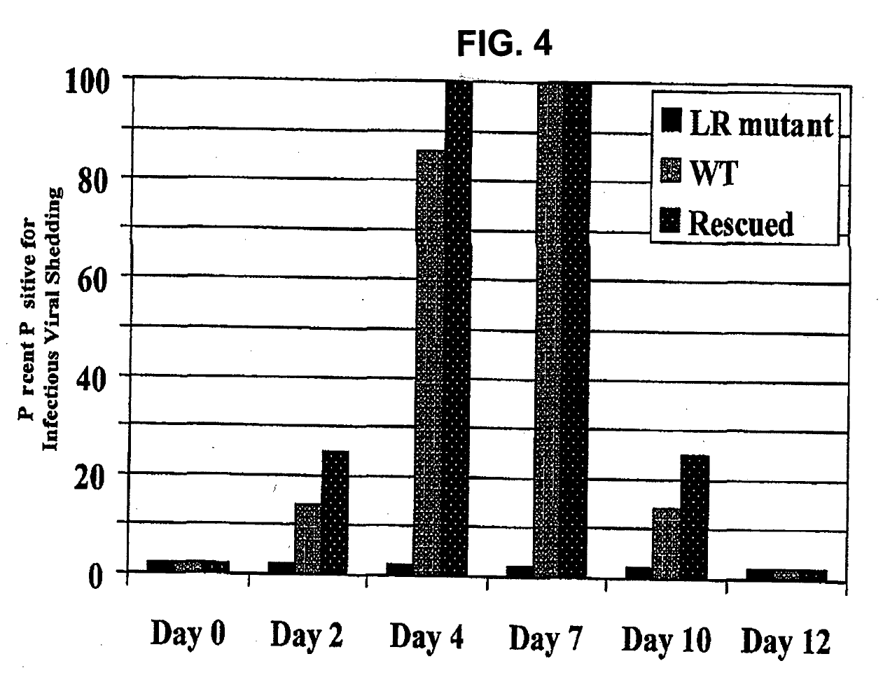

Infectious virus was not detected in calves latently infected with the LR mutant following DEX treatment to initiate reactivation (FIGS. 3 and 4). No changes in virus-specific antibodies were detected during the course of reactivation. An increase in virus-specific antibodies is a sensitive method to detect reactivation from BHV-1 (Jones et al., 2000) and HSV-1 (Perng et al., 1999). Furthermore, multiple injections of DEX were used to ensure that reactivation occurred. By using a similar protocol, a modified live vaccine strain of BHV-1 that is severely attenuated has been reactivated (Jones et al., 2000). It is generally accepted that the amount of viral DNA in TG of HSV-1-infected small animal models plays a role in the efficiency of reactivation (Jones 1998; Wagner and Bloom, 1997), implying there is a similar mechanism for BHV-1. As discussed above, calves infected with the wt, but not the LR mutant, have neurons with high levels of viral DNA and reactivate efficiently.

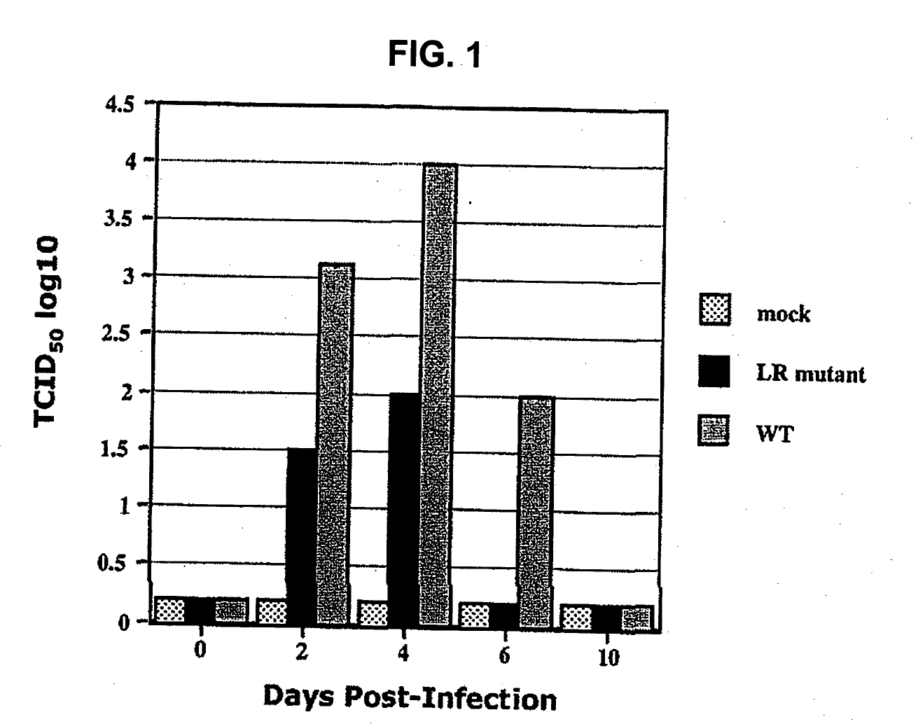

In general, HSV-1 LAT has been shown to be important, but not required, for latency reactivation in rabbits and mouse models (Jones 1998; Wagner and Bloom, 1997). The results reported herein show that wt expression of LR gene products was required for the latency reactivation cycle in calves when reactivation was initiated by DEX. One possible explanation for this is that the LR gene is more important than LAT for the latency reactivation. BHV-1 lacks several genes contained in the HSV-1 genome, which mediate pathogenesis and/or latency: the 34.5 gene, for example (Schwyzer and Ackermann, 1996). The 34.5 gene plays a crucial role in neurovirulence by inhibiting antiviral functions of the interferon-inducible double-stranded RNA-dependent protein kinase R (Chou et al., 1995; Leib et al., 2000). 34.5-null mutants have reduced pathogenesis in rabbits and mice, in large part because of poor growth properties in the eyes and TG (Perng et al., 1995). Since LR gene products play a role in ocular growth of BHV-1 in calves (Inman et al., 2001B) and lower levels of infectious virus were detected in TG of calves acutely infected with the LR mutant (FIG. 1), they have additional properties that have not been described for HSV-1 LAT. The LR gene restores spontaneous reactivation to a McKrae LAT-null mutant (dLAT2903), demonstrating that LAT and LR gene products have common functional properties that are necessary for efficient reactivation from latency (Perng et al., 2002). In addition, the LR gene enhanced the ability of dLAT2903 to kill mice during acute infection and induce recurrent eye disease in latently infected rabbits, underscoring our hypothesis that LR gene products have expanded roles during the latency reactivation cycle and even pathogenesis when compared to LAT.

Method of Use: Vaccines for Bovine Herpesvirus 1

Novel BHV-1 vaccines of the present invention are based on the discovery that: 1) the LR gene of BHV-1 is not required to establish acute intranasal infection; and 2) the LR gene is essential for reactivation of BHV-1 from latency. The present invention accordingly seeks to provide novel vaccines based on live BHV-1, attenuated by mutation in the latency-related gene, which elicit an immune response in the host, however do not reactivate from latency.

BHV-1 strains comprising a mutation in the LR gene are provided. The mutation can be obtained by means well known in the art, such as but not limited to, genetic engineering techniques, chemical synthesis, mutant selection techniques, or the method described in Example 2. Any methods which do not interrupt bICP0 gene expression, yet interrupt LR gene expression, may be employed.

In a preferred method, the mutant oligonucleotide is inserted into the Cooper strain of BHV-1 (ATCC No. VR-864). In addition to the LR mutation, one or more additional deletions or modifications to the coding sequences of a glycoprotein are preferably included to further attenuate the Cooper Strain of BHV-1. Examples of BHV-1 glycoprotein genes that can be deleted, or modified by insertion, substitution or otherwise are: glycoprotein C, glycoprotein D, glycoprotein E, glycoprotein G, glycoprotein H, or glycoprotein I. Although this is a preferred embodiment, other means to arrive at an LR-null mutant may be used in the practice of the invention. Further, a mutant LR gene could be inserted into other commercially available strains of BHV-1, for example, without limitation, BovaShield (Pfizer, Inc.) or the temperature sensitive mutant described in Jones et al. (2000). In one preferred embodiment, the BHV-1 strain is less virulent than the Cooper strain of BHV-1. Further mutations may be made using the plasmid (pBlueL/mLAT) (Inman et al., 2001B). Briefly, plasmid (pBlueL/mLAT) can be constructed as follows: 825 base pairs (bp) of the BHV-1 HindIII L fragment that is directly upstream of the LR promoter (D fragment) are cloned into pBlueBacHisA (InVitrogen, Carlsbad, Calif.). pBR322-HindIII L-fragment contains the HindIII L fragment of BHV-1 (Cooper Strain) and this plasmid is digested with NheI. The resulting products are treated with Mung Bean exonuclease (New England BioLabs) to blunt the ends for ligation of BamHI linkers. After phenol and chloroform extraction, the DNA is digested with HindIII and then BamHI. The products are electrophoresed on an agarose gel and the 825 bp product isolated. The 825 bp product containing a 5′ BamHI site and a 3′ Hind III site is ligated into the pBlueBacHis vector digested with BamHI plus HindIII (resulting plasmid designated herein as pBlueL). A fragment containing the entire LR promoter and coding region (1940 bp) is cloned into the HindIII and SalI sites of pBlueL (resulting plasmid designated herein as pBlueL/LAT). The PstI fragment (1-981 nt) is excised from pBlueL/LAT and cloned into pBlueBacHis vector. This sub-cloning is performed because there are 3 SphI sites in the coding region of the LR gene (781, 812 and 1777 nt). The SphI fragment (781-812 NT) is excised and the mutant oligonucleotide inserted. The mutated PstI fragment is then cloned back into the original PstI digested pBlueL/LAT and the resulting construct (designated herein as pBlueL/mLAT). Restriction enzyme mapping and DNA sequencing can be used to determine proper orientation of the PstI fragment. All cloning procedures (restriction digests, ligations, CIP treatment, etc.) are performed by standard procedures. All BHV-1 fragments can be obtained from the Cooper strain of BHV-1 (ATCC VR-864).

The coding sequences for the glycoprotein genes listed above are known, and primers can be designed to amplify these genes. Deletions can be generated using standard molecular biology techniques such as restriction enzyme digestions, PCR mediated mutagenesis, or site directed mutagenesis. These deletions are introduced back into the Cooper strain using homologous recombination, as described in Example 2 for constructing the LR mutant.

Bovine epidermal cells are cotransfected with 6 ug pBlueL/mLAT, 2 ug of a plasmid encoding HSV-1 ICP0, and 2 ug of viral DNA using Superfect (Qiagen) as previously described (Inman et al., 2001B; Inman et al., 2002). Sixteen hours after transfection, cells are split 1:2, incubated for 16 additional hours, and then overlaid with 0.7% Seaplaque agarose. When visible plaques appear (3-4 days after infection); each plaque is isolated, propagated in MDBK cells, and screened by PCR for the mutant oligonucleotide insert. PCR is performed on the extracted DNA using primer 4 (p4) (nt 873 5′CGTGTATTTGCGACCCCCAGCCT3′) and primer 5 (p5) (nt 596 5′GCCAGACCAAACCCCCCGCA3′). After a hot start, each cycle consists of 95° C. for 1 min., 60° C. for 1 min. and 72° C. for 2 min. (30 cycles total). To ensure complete elongation of amplified products, the reaction is incubated at 72° C. for an additional 10 minutes. Products are digested with EcoRI, electrophoresed on a 2% agarose gel, and the DNA visualized by staining with ethidium bromide.

Isolation of intact BHV-1 viral DNA of the live attenuated vaccine strain can be performed as previously described (Inman et al., 2001B; Inman et al., 2002). Briefly, MDBK cells can be infected with the BHV-1 strain obtained from Pfizer at a multiplicity of infection of approximately 10. The clarified lysate can be pelleted using a 30% sucrose/TE cushion (25,000 rpm. for 2 h in a Beckman Lt-65 using an SW28 rotor at 4° C.). Virions can be disrupted with SDS, RNAse treatment, followed by proteinase K, and extraction with phenol/chloroform/isoamyl alcohol (50:48:2). The integrity and quantity of viral DNA is determined by 1% agarose gel electrophoresis. Stocks of virus can be prepared in MDBK cells as described previously (Inman et al., 2001B; Inman et al., 2002; Jones et al., 2000).

Vaccination of animals with the live attenuated vaccines of the present invention can be conducted by means well known in the art (Jones et al., 2000). Briefly, for intranasal instillation, calves can be inoculated with approximately 6.5 log₁₀ TCID₅₀/ml of the respective modified live vaccine strains. For IM vaccination, the modified live vaccine can be injected into the flank (caudal muscle mass) or the neck (brachiocephalicus muscle) using 6.5 log₁₀ TCID₅₀/ml of the virus. An 18 gauge needle (1.5 inch) can be used for all IM injections, and the injection can contain approximately 0.5 ml. Because the attenuated virus can grow efficiently in the nasal cavity a robust BHV-1 specific antibody response occurs in infected calves. However, the clinical symptoms and disease are not significant. The construction of a LR mutant and route of vaccination provided above are exemplary only, and any suitable means known in the art may be used in the practice of the present invention

Method of Use: Vaccines for Non-Herpes Viral and Bacterial Pathogens

Novel vaccines based on live BHV-1 attenuated by mutation in the latency-related gene such that the virus will not reactivate from latency, may be used to provide vaccines for a variety of viral and bacterial pathogens. Recombinant nucleic acids may be inserted into any region of the viral genome of attenuated BHV-1 strains of the present invention which is non-essential for infection of the host, using methods known in the art. For example, without limitation, two such approaches are: 1) insertion of recombinant nucleic acid form the pathogen in the mutated LR gene locus such that expression is driven by the LR promoter to which it is operably linked; and 2) insertion of recombinant nucleic acid from the pathogen gene into a glycoprotein locus of an LR mutant virus, and the expression of the pathogen nucleic acid driven by a foreign promoter to which it is operably linked.

For the first approach, the pBlueL/mLAT plasmid (Inman et al., 2001B) is modified to contain a unique PacI restriction enzyme site at nucleotide 781 of LR sequences (the first SphI site that was used to insert the mutant oligo). Genes from these pathogens are cloned and PacI sites added to the termini of these fragments. This approach permits expression of the foreign gene operably linked to the LR promoter in the context of the BHV-1 genome.

For the second approach, recombinant nucleic acid of other pathogens (or homologous nucleic acid of other BHV-1 antigens) are inserted into the glycoprotein C (gC) or glycoprotein E (gE) locus in the context of the LR mutant virus. The gC and gE loci are chosen because these genes are not important for growth of the virus in cultured cells, thus readily permitting construction of recombinant viruses. The human cytomegalovirus promoter can be used to drive expression of the respective genes from other pathogens as this promoter is expressed efficiently during herpes simplex virus type 1 (HSV-1) acute infection, and thus can be expected to be expressed efficiently during BHV-1 productive infection.

Pathogens that are candidates for this approach include, without limitation: bovine viral diarrhea virus (BVDV), bovine respiratory virus (BRSV), bovine corona viruses, and bacterial strains commonly associated with “Shipping Fever” (Pasteurella haemolytica, Pasteurella multocida, and Haemophilus somnus for example). For the viruses listed, structural proteins are used for foreign expression. For the bacterial pathogens, immunogenic surface proteins are used.

Following vaccination of calves, a specific immune response is induced against BHV-1 and the gene from the respective foreign pathogen. This immune response is determined by measuring antibody responses.

### EXAMPLES

The present invention is further described in the following examples, which are offered by way of illustration and are not intended to limit the invention in any manner. Standard techniques well known in the art or the techniques specifically described below are utilized.

### Example 1

### Materials and Methods

A. Virus and Cells

Bovine kidney cells (MDBK; ATCC CCL-22) were plated at a density of 5×10⁵ per 100-mm² plastic dish in Earle's modified medium supplemented with 5% fetal bovine serum (FBS), penicillin (10 U/ml), and streptomycin (100 μg/ml).

The Cooper strain of BHV-1 (wt virus) (ATCC VR-864) was obtained from the National Veterinary Services Laboratory, Animal and Plant Health Inspection Services, Ames, Iowa. A complete description of the LR mutant virus has been previously described (Inman et al., 2001B incorporated in full herein, by reference). Briefly, the LR mutant virus was developed by replacing wt (Cooper strain) LR gene sequences with an oligonucleotide that contains a unique EcoRI restriction site and three stop codons in each reading frame (FIG. 2). In transiently transfected cells, a plasmid with this mutation does not express detectable levels of the LR protein, but does express LR-RNA (Ciacci-Zanella et al, 1999). Viral stocks were prepared by infecting MDBK cells with a multiplicity of infection (MOI) of 0.001 from a plaque-purified virus. Virus was titrated on MDBK cells by using 10-fold dilutions and determining the 50% tissue culture infectious dose (TCID₅₀) or PFU.

B. Animal Experiments.

BHV-1-free crossbred calves (˜250 kg) were randomly assigned and housed in isolation rooms to prevent cross contamination. Calves were anesthetized with Rompun (approximately 1 mg/kg of body weight; Bayer Corp., Shawnee Mission, Kans.). Calves were then inoculated with 1 ml of a solution containing 10⁷ PFU of the indicated virus per ml in each nostril and eye, without scarification, for a total of 4×10⁷ PFU per animal as described previously (Schang and Jones, 1997; Winkler et al., 1999; Winkler et al., 2000). Experiments with animals were performed in accordance with the American Association of Laboratory Animal Care guidelines. At 60 days postinfection (dpi) (latency), calves were injected intravenously with 100 mg of DEX as described previously (Winkler et al., 2000). Additional intramuscular injections (25 mg) of DEX were given at 2 and 4 days after the initial intravenous injection of DEX to ensure that reactivation occurs. We have previously demonstrated that multiple injections of DEX enhance shedding of virus (Jones et al., 2000). Calves were housed under strict isolation containment and given antibiotics before and after BHV-1 infection to prevent secondary bacterial infection.

Nasal swabs, ocular swabs, and serum samples were taken at the designated times. Nasal and ocular swabs were stored at −80° C. in 2 ml of tissue culture medium supplemented with 10 μg of amphotericin B per ml (Fungizone) and 45 μg of gentamicin per ml. Samples were thawed quickly in a 37° C. water bath, vortexed, and centrifuged (1,500×g for 10 min). All titrations were performed with 10-fold serial dilution and plated in quadruplicate.

Virus was isolated from TG by mincing 0.5 g of tissue, suspending the tissue in 9 ml of Dulbecco's modified Eagle's medium (DMEM), and homogenizing the tissue with a tissue grinder (Polytron, Switzerland). One milliliter of fetal bovine serum was added, and the homogenate was subjected to three freeze-thaw cycles with a dry ice-ethanol bath. After the last cycle, the homogenate was centrifuged at 2,000 rpm (Jouan CR412 centrifuge) for 30 min at 4° C. The supernatant from the TG homogenate was subsequently used to infect MDBK cells. The TG supernatant (125 μl) was added to 500 μl of medium (1:5 dilutions), and then 1:5 serial dilutions were made. One hundred microliters of each dilution was added in quadruplicate to a 96-well plate. One hundred microliters of MDBK cells (10⁵ cells) was added to each well. After 4 days of incubation, cells were fixed and then stained with formaldehyde-bromophenol blue. Virus titers were measured with the 50% end point assay as described previously (Inman et al., 2001B).

C. Nucleic Acid Tissue Extraction

RNA and DNA extractions from MDBK cells or TG were performed as previously described (Jones et al., 2000; Schang and Jones, 1997).

D. Reverse Transcription of RNA to Obtain cDNA

Reverse transcription (RT) was performed essentially as previously described (Jones et al., 2000). Briefly, 4 μg of RNA was treated with DNase, and after inactivation, the RNA was reverse transcribed with random hexamers as primers. As a control for DNA contamination in the RNA samples, DNase-treated RNA was mixed with the RT reaction mix lacking reverse transcriptase. Two microliters was used for PCR.

E. PCR

PCR was performed on the extracted DNA and synthesized cDNA with the indicated primer pairs p4 (nucleotide [nt] 873, 5′CGTGTATTTGCGACCCCCAGCCT3′ (SEQ ID NO:1)) and p5 (nt 596, 5′GCCAGACCAAACCCCCCGCA3′ (SEQ ID NO:2)) (Inman et al., 2001B; Schang and Jones, 1997). Actin, L3B, and gC primers have been described previously (Hossain et al., 1995; Inman et al., 2001B; Jones et al., 2000; Schang and Jones, 1997). The gC forward primer is 5′ AAAGCCCCGCCGAAGGAG (SEQ ID NO:3) (bp 550 of the BHV-1 gC gene). The gC reverse primer is 5′ TACGAACAGCAGCACGGGC (SEQ ID NO:4) (bp 756 of BHV-1 gC gene). The forward bovine growth hormone (gH) primer is 5′ GCTTTCGCCCTGCTCTGCC (SEQ ID NO:5) (bp 994 of the bovine growth hormone gene). The reverse gH primer is 5′ TCCTGCCTCCCCACCCCTA (SEQ ID NO:6) (bp 1155 of the bovine growth hormone gene). After a hot start, each cycle consisted of 95° C. for 1 min, 60° C. for 1 min, and 72° C. for 2 min (30 cycles total). To ensure complete elongation of amplified products, the reaction was incubated at 72° C. for an additional 10 min. For some studies, the PCR products were digested with EcoRI, electrophoresed on a 2% agarose gel, and the DNA was visualized by staining with ethidium bromide.

F. Direct Fluorescence Assay for the Detection of BHV-1

For the direct fluorescence assay (FA), a 24-well plate of MDBK cells was infected with clarified viral lysate. Twenty-four hours later, cell monolayers were fixed for 5 min with ice-cold methanol-acetone (50:50) and then allowed to dry. A 1:50 dilution of direct conjugate (anti-BHV-1-specific antibody-fluorescein isothiocyanate [FITC] conjugate; American BioResearch, Milton, Tenn.) was added to the fixed cells and incubated at 37° C. for 30 min. The monolayers were washed twice with phosphate-buffered saline. The presence of virus was visualized with an FITC filter.

G. BHV-1-Specific Neutralizing Antibodies

The Veterinary Diagnostic Service, University of Nebraska, Lincoln, measured neutralizing antibody titers with the Cooper strain as the stock virus, using standard materials and methods.

H. In Situ Hybridization

DNA probes specific for BHV-1 gC, ribonucleotide reductase, bICP0, and the LR gene were used. Labeling of the probes and hybridization steps were performed as previously described (Winkler et al., 1999; Winkler et al., 2000). After hybridization, slides were washed twice in 4×SSC (1×SSC is 0.15 M NaCl plus 0.015 M sodium citrate) for 5 min at room temperature, once in 2×SSC at 40° C., once in 0.5×SSC at 40° C., twice in 2×SSC at room temperature, twice in 0.5×SSC at room temperature, and once in buffer I (100 mM maleic acid, 150 mM NaCl [pH 7.5]). The conjugate antibody step and reaction development were also performed as previously described (Winkler et al., 1999; Winkler et al., 2000).

### EXAMPLE 2

### Construction of a BHV-1 LR Mutant Virus

To determine whether BHV-1 LR gene products play a role in virus growth and/or latency, a BHV-1 LR mutant virus that contains stop codons near the 5′ terminus of the LR transcript was constructed and tested in cultured cells and calves.

The LR gene is transcribed antisense with respect to the immediate-early (IE) and early (E) gene transcript (IE/2.9 and E/2.6) that encodes bICP0. The lytic start site for the LR RNA is at nt 724 (Ahmed et al., 2002; Hariharan et al., 1993), and the first in-frame ATG for LR open reading frame 2 (ORF2) is at nt 783 to 785, whereas the stop site for bICPO is at nt 956 (LR numbers) (Devi-Rao et al., 1994; Rock et al., 1987A; Rock et al., 1987B; Sawtell, 1997), which complicates construction of an LR mutant virus. The cis-acting sequences that regulate poly(A) addition for the transcript that encodes bICP0 are also near sequences that contain the LR gene TATA box. This prevented insertion of a reporter gene near the start site of LR gene expression or an extensive deletion of LR gene sequences. Consequently, we inserted three stop codons to prevent LR protein expression in all three reading frames. This mutation was also designed to allow for WT levels of bICP0 expression. The entire promoter and coding region of the LR gene was cloned into the pBlueBacHisA vector as described previously (Inman et al., 2001B). A total of 825 bases from the adjacent HindIII L fragment (Inman et al., 2001A) were cloned upstream of the LR promoter to ensure that efficient homologous recombination occurred. The LR sequences between the two SphI sites (nt 781 to 812) were replaced with the mutant oligonucleotide. The mutant oligonucleotide contains the first in-frame ATG of ORF2, a unique EcoRI restriction site to facilitate screening, and three stop codons that are in each reading frame. In transiently transfected cells, this LR mutant gene construct expresses the LR RNA, but the protein detected by a peptide antibody directed against the N terminus of LR ORF2 (P2) is not detected (Carter et al., 1989; Hariharan et al., 1993).

BHV-1 DNA was extracted from infected cells, and its integrity was examined by agarose gel electrophoresis. BHV-1 DNA is not very infectious when transfected into cultured bovine epithelial cells. Efficient plaque formation was not observed at 14 days posttransfection, a time when cells were lifting off the plates. When BHV-1 DNA and plasmids encoding bICPO (Inman et al., 2001C) or HSV-1 ICPO were cotransfected into bovine cells, efficient plaque formation was consistently observed 48 h after transfection. A plasmid expressing HSV-1 ICPO was used for these studies because bICPO sequences overlapped the LR mutant region, and thus we were concerned this might reduce the efficiency of homologous recombination.

The viral genome was cotransfected into bovine epithelial cells with HSV-1 ICPO and the plasmid containing the mutant oligonucleotide (pBlueL/mLAT). Plaques were isolated and screened for insertion of the mutant oligonucleotide sequence by PCR using the p4 and p5 primers. The amplified products were then digested with EcoRI. If homologous recombination between the LR mutant plasmid and the viral genome occurred, two bands (105 and 193 bp) would be observed following digestion with EcoRI. WT virus yielded a single band (298 bp) as expected. After the potential LR mutants were subjected to three rounds of plaque purification, the same banding pattern was observed, demonstrating that the mutant virus from a plaque was not contaminated with WT virus and was stable. Several plaques containing the mutant were selected and plaque purified two more times to ensure they were not contaminated with WT virus.

To ensure that a resulting phenotype was not due to secondary site mutations, a rescued virus was constructed (LR rescued virus). The LR mutant viral genome was cotransfected with the WT LR gene cloned into pBlueBacHisA (pBlueL/LAT) and a plasmid encoding HSV-1 ICPO into bovine cells. The p4 and p5 primers were used to identify amplified products that were not digested by EcoRI, which was indicative of the LR WT gene. Viral sequences encompassing the manipulated regions of the LR gene in the LR mutant and LR rescued virus were sequenced and contained the expected sequences.

### EXAMPLE 3

### Analysis of the BHV-1 LR Mutant Virus in MDBK Cells

Infection of MDBK cells with BHV-1 Cooper strain produces visible cytopathic effects by 7 to 10 h p.i. followed by efficient plaque formation. IE gene expression can be detected within 1 to 2 h p.i. Although not statistically significant, growth curves suggested that the mutant released virus slightly faster from MDBK cells early in infection at an MOI of 1. However, the end point titers were consistently the same. At an MOI of 5, there were no differences in the titers of cell-associated and released virus. It was consistently observed that resting cells or cells that were too confluent did not yield as much virus as actively growing cells. However, the LR mutant and WT virus had similar growth properties in MDBK cells. LR mutant, LR rescued, and WT viruses also had similar growth properties in rabbit epidermal (CCL-68; ATCC), rabbit lung (CCL-193; ATCC), rabbit skin fibroblasts (CRL-1414; ATCC), and bovine epidermal cells.

The LR RNA is antisense to the IE and E transcript that is translated into bICP0 (Jones 1998), suggesting bICP0 expression could be altered by the mutation within the LR gene. Since bICP0 is essential for productive viral replication (Fraefel et al., 1994; Inman et al., 2001C; Koppel et al., 1996), expression of bICP0 in the WT, LR rescued, and LR mutant viruses following infection of MDBK cells were compared. These studies demonstrated that expression of bICP0 in MDBK cells infected with the LR mutant was at least as high as in those infected with the WT or the LR rescued virus.

### EXAMPLE 4

### Construction of CJLAT

The plasmid p1658 contains two HSV-1 DNA restriction fragments. One restriction fragment (HpaI-EcoRV) corresponds to the region from 1800 to 161 nucleotides upstream of the LAT start site (LAT nucleotides −1800 to −161). The other restriction fragment (HpaI-MluI) corresponds to LAT nucleotides 1667 to 2850. A unique PacI restriction site was cloned between these restriction fragments (i.e., between LAT nucleotides −161 and +1667). PacI linkers were added to the complete BHV-1 LR gene DNA, which was then cloned into the above unique PacI restriction site of p1658. The LR gene was a 1.9-kb HindIII-SalI fragment derived from the HindIIID fragment of BHV-1 strain Cooper (BHV-1 nucleotides 1 to 1941) that contains the LR promoter and the known coding sequences (Kutish et al., 1990). The resulting plasmid, CJLATP1658, was cotransfected with infectious dLAT2903 genomic DNA into RS cells as we previously described for construction o other HSV-1 mutants (Perng et al., 1994; Perng et al., 1996). After homologous recombination between the HSV-1 DNA flanking the BHV-LR DNA in the plasmid and the genomic DNA flanking the LAT deletion in dLAT2903, the cotransfection mix was plated on RS cells, and viral plaques were isolated. Individual plaques were analyzed by restriction digestion and Southern analysis. In the final chimeric virus, CJLAT, the core LAT promoter and the region of the LAT gene corresponding to the first 1.7-kb of the primary LAT transcript of HSV-1 McKrae has been replaced by the BHV-1 LAT gene. CJLAT was triple plaque purified and its genomic structure was confirmed by additional restriction digestion-Southern analysis.

### EXAMPLE 5

### Growth of LR Mutant Virus in TG of Acutely Infected Calves

BHV-1 LR mutant virus containing stop codons near the 5′ terminus of the LR transcript (LR mutant) where constructed, as previously described (Inman et al., 2001B). The LR mutant grew to similar titers relative to wt or LR-rescued virus in the nasal cavity of calves and cultured bovine cells. However, the LR mutant shed approximately 4 logs less infectious virus from eyes of acutely infected calves compared to wt or LR-rescued virus, suggesting the LR gene plays a role in acute virus growth in certain bovine tissue.

Presence of infectious virus. To test whether calves infected with the LR mutant contained different amounts of infectious virus in TG compared to calves infected with wt BHV-1, calves were infected with the respective viruses, TG were harvested at different times after infection, and infectious virus was measured in TG homogenates. Levels of infectious virus in TG homogenates were measured by incubating the supernatants with MDBK cells. Cultures that produced cytopathic effects were also subjected to FA with BHV-specific antisera as an independent verification that cytopathic effects were due to virus infection and not nonspecific toxic effects of the TG homogenates. At 2 and 4 dpi, calves infected with the LR mutant contained 10- to 100-fold less virus in TG homogenates compared to calves infected with wt virus (FIG. 1). Infectious virus was not detected in TG of calves infected with the LR mutant on 6 dpi. FA-positive samples were detected at 6 dpi in LR mutant-infected calves and 10 dpi in calves infected with the wt (data not shown), but the amount of virus was too low for titration.

Presence of LR mutant genome. To verify that the LR mutant genome was present in TG of calves infected with the LR mutant, PCR analysis was performed. DNA was prepared from calves infected for 6 or 10 dpi and screened for the presence of the mutant by PCR with the p4 and p5 primers. Amplified products were then digested with EcoRI. Calves infected with the LR mutant contained two bands (105 and 193 bp) that were detected following digestion with EcoRI. The presence of wt virus yields a single band of 298 bp, while LR mutant virus yields two bands migrating at 105 and 193 bp. The LR mutant plasmid and a plasmid with the wt LR gene were used as positive controls to show the positions of the expected products. PCR of representative samples from calves infected for 6 or 10 days demonstrates that only the inoculated virus was detected in the TG. In summary, this study demonstrated that, during acute infection, lower levels of infectious virus were present in TG of calves infected with the LR mutant.

Analysis of BHV-1 DNA in calves infected with the LR mutant or wt DNA. In situ hybridization was performed with probes that specifically bind to four viral genes (Winkler et al., 1999; Winkler et al., 2000). Thin sections were prepared from TG of calves infected with the LR mutant or wt virus at 10 (end of acute infection or establishment of latency) or 60 (latency) dpi. In situ hybridization was performed as described in Example 1. In situ-positive neurons in TG were consistently detected in thin sections prepared from calves infected with wt virus at 10 and 60 dpi. We estimated that less than 5% of the total neurons contained viral DNA, as judged by in situ hybridization. In contrast, DNA-positive neurons were not detected by in situ hybridization with TG thin sections prepared from calves infected with the LR mutant for 10 or 60 days after infection, This study suggested that neurons with high copies of viral DNA were not present in TG of calves infected with the LR mutant.

To further compare the levels of viral DNA in TG of calves infected with the wt virus or the LR mutant, a semiquantitative PCR was performed as described in Example 1. PCR primers were used to detect BHV-1 gC DNA and bovine growth hormone (gH). PCR products were electrophoresed on 2% agarose gels, and the DNA was stained with ethidium bromide. DNA prepared from: TG of a mock-infected calf; MDBK cells infected with BHV-1 (MOI=1) for 24 h; and from BHV-1 virions (purified) were included as controls. For each DNA sample, 1 μg of DNA was used for PCR. 20 ng of purified viral DNA was used for PCR.

We focused on samples prepared from latently infected calves 60 days after infection (60 dpi) or during the early stages of reactivation (24 and 48 h after single intravenous injection with DEX). As expected, wt viral DNA was readily detected in TG of latently infected calves and at 24 or 48 h after DEX treatment. In contrast, TG prepared from calves latently infected with the LR mutant did not contain detectable levels of viral DNA. At 24 h after DEX treatment, neither of two calves latently infected with the LR mutant contained detectable levels of viral DNA. At 48 h after DEX treatment, one of three calves latently infected with the LR mutant contained detectable levels of viral DNA. Under the conditions of this PCR assay, we were able to detect approximately 200 to 400 copies of viral DNA per μg of total DNA. In summary, two independent assays (in situ hybridization and PCR) demonstrate that calves latently infected with the LR mutant contained less viral DNA in TG.

### EXAMPLE 6

### LR-RNA Expression in Calves Latently Infected with the LR Mutant

In theory, the LR mutant should express LR-RNA, but not proteins because of the three stop codons (Inman et al., 2001B). RNA was extracted from TG of infected calves or MDBK cells as described in Example 1. cDNA was generated with random primers and PCR was performed with specific primers. Primers that amplify a segment of the β-actin transcript flank an intron, which allows for detection of contaminating DNA as previously described (Inman et al., 2001B). L3B primers were used to detect the LR transcript. Primers that amplify gC RNA were used to verify that calves were in the latent phase of infection. When reverse transcriptase was omitted during cDNA synthesis, no PCR product was detected. In productively infected bovine cells, the LR mutant expressed abundant levels of LR-RNA. LR-RNA expression was examined in TG of calves infected with the LR mutant at 60 dpi (latency). RT-PCR was performed with primers that specifically amplify LR-RNA (L3B) as described previously (Ciacci-Zanella et al, 1999; Hossain et al., 1995; Jones et al., 2000). LR-RNA was detected in two of two calves infected with the LR mutant and, as expected, in two of two calves infected with the wt at 60 dpi. gC RNA was not detected by RT-PCR in latently infected calves, demonstrating that high levels of productive infection were not occurring in TG during latency (60 days after infection).

### EXAMPLE 7

### Effect of DEX on Reactivation of the LR Mutant

Treatment of latently infected rabbits (Rock et al., 1992) or calves (Jones et al., 2000; Sheffy and Davies, 1972) with DEX consistently reactivates BHV-1 from latency. Consequently, virus shedding occurs in ocular or nasal cavities, and virus-specific antibodies increase. A preliminary study was performed with two calves latently infected with the LR mutant, two calves infected with wt virus, and two calves infected with the LR-rescued virus. Following a single intravenous injection of DEX (100 mg), we detected reactivation in calves latently infected with wt or the LR-rescued virus, but not the two calves infected with the LR mutant, suggesting the LR mutant was not capable of reactivating. To confirm this result, a larger study was performed with an initial intravenous injection of DEX (100 mg) followed by two intramuscular injections of DEX (25 mg) to ensure that reactivation occurred efficiently. Our previous studies demonstrated that multiple injections of DEX allowed efficient reactivation of a temperature-sensitive mutant of BHV-1, which is used as a modified live vaccine and is severely attenuated (Jones et al., 2000).

Neutralizing antibody titers were measured after DEX treatment to determine if reactivation occurred. Serum was collected from calves at the indicated times and stored at −20° C. until tested. Standard testing was performed with constant amounts of virus (Cooper strain) and twofold serial dilutions of the serum. The Veterinary Diagnostic Services, University of Nebraska, Lincoln, performed the assay. As expected, a dramatic increase in virus-specific antibodies was detected in animals infected with wt or LR-rescued virus after DEX-induced reactivation (FIG. 3), indicating that virus replication and shedding occurred. A five- to sixfold increase in antibody titers was detected, which is consistent with reactivation from latency. In contrast, an increase in virus-specific antibodies was not observed when calves latently infected with the LR mutant were treated with DEX.

To confirm the neutralizing antibody results, ocular and nasal swabs were collected from each calf at the designated times after treatment with DEX. Infectious virus in swabs was detected by inoculating MDBK cells with a 1:20 dilution of the swab medium solution. FA was used to confirm the presence of infectious BHV-1 in cultures containing cytopathic effects. As expected, during latency, wt, rescued, or LR mutant virus was not detected in swabs on the day of DEX treatment (day 0). At 4 and 7 days after DEX treatment, infectious virus was detected in swabs obtained from six of seven and seven of seven calves latently infected with wt virus (FIG. 4). At 4 and 7 days after DEX treatment, infectious virus was detected in four of four calves infected with the rescued virus. At these time points, infectious virus was detected in nasal and ocular swabs of calves shedding wt and LR-rescued virus. To simplify the data, the ocular and nasal results were combined. In sharp contrast, infectious virus was not detected in seven of seven calves latently infected with the LR mutant at any time point tested. Infectious virus was not detected in nasal or ocular swabs of any calf after 10 days postreactivation. These studies demonstrated that the LR mutant did not reactivate from latency following multiple injections of DEX.

### DEPOSIT INFORMATION

The live, attenuated BHV-1 strain has been deposited with the American Type Culture Collection (ATCC), 10801 University Boulevard, Manassas, Va. 20110-2209, USA, on ______, in accordance with the Budapest Treaty and has been accorded Accession Number ______.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and sub-combinations are of utility and may be employed without reference to other features and sub-combinations. This is contemplated by and is within the scope of the claims.

### LIST OF REFERENCES

Ahmed et al, 2002. Regions of the herpes simplex virus type 1 latency-associated transcript that protect cells from apoptosis in vitro and protect neuronal cells in vivo. J. Virol. 76:717-729.

Block et al. 1990. A herpes simplex virus type 1 latency-associated transcript mutant reactivates with normal kinetics from latent infection. J. Virol. 64:3417-3426. (Erratum, 64:4603.)

Bloom et al., 1996. A 348-base-pair region in the latency-associated transcript facilitates herpes simplex virus type 1 reactivation. J. Virol. 70:2249-2259.

Bratanich et al., 1992. The latency-related gene of bovine herpesvirus 1 inhibits the activity of immediate-early transcription unit 1. Virology 191:988-991.

Carter et al., 1989. Inhibition of T-lymphocyte mitogenic responses and effects on cell functions by bovine herpesvirus 1. J. Virol. 63:1525-1530.

Chou et al., 1995. Association of a Mr 90,000 phosphoprotein with protein kinase PKR in cells exhibiting enhanced phosphorylation of translation initiation factor eIF-2 alpha and premature shutoff of protein synthesis after infection with gamma 134.5-mutants of herpes simplex virus 1. Proc. Natl. Acad. Sci. USA 92:10516-10520.

Ciacci-Zanella, et al., 1999. The latency-related gene of bovine herpesvirus 1 inhibits programmed cell death. J. Virol. 73:9734-9740.

Coffin et al., 1998. The herpes simplex virus 2-kb latency associated transcript (LAT) leader sequence allows efficient expression of downstream proteins which is enhanced in neuronal cells: possible function of LAT ORFs. J. Gen. Virol. 79:3019-3026.

Devi-Rao et al., 1994. Herpes simplex virus type 1 DNA replication and gene expression during explant-induced reactivation of latently infected murine sensory ganglia. J. Virol. 68:1271-1282.

Devireddy and Jones, 1998. Alternative splicing of the latency-related transcript of bovine herpesvirus 1 yields RNAs containing unique open reading frames. J. Virol. 72:7294-7301.

Drolet at al., 1998. The region of the herpes simplex virus type 1 LAT gene involved in spontaneous reactivation does not encode a functional protein. Virology 242:221-232.

Fraefel et al., 1994. Identification and zinc dependence of the bovine herpesvirus 1 transactivator protein BICP0. J. Virol. 68:3154-3162.

Goding, 1986. Monoclonal Antibodies: Principles and Practice, 2d ed. (Academic Press).

Griebel et al., 1990. The interaction between bovine herpesvirus type 1 and activated bovine T lymphocytes. J. Gen. Virol. 71:369-377.

Griebel et al., 1987A. T lymphocyte population dynamics and function following a primary bovine herpesvirus type-i infection. Viral Immunol. 1:287-304.

Griebel et al., 1987B. Bovine peripheral blood leukocyte subpopulation dynamics following a primary bovine herpesvirus-1 infection. Viral Immunol. 1:267-286.

Hariharan et al., 1993. Down regulation of murine MHC class I expression by bovine herpesvirus 1. Viral Immunol. 6:273-384.

Harlow and Lane, 1988. Antibodies: A Laboratory Manual (Cold Spring Harbor Laboratory, Cold Spring Harbor, N.Y.).

Hinkley, et al. 1998. Bovine herpesvirus-1 infection affects the peptide transport activity in bovine cells. Virus Res. 53:91-96.

Ho and Mocarski, 1988. Beta-galactosidase as a marker in the peripheral and neural tissues of the herpes simplex virus-infected mouse. Virology 167:279-283.

Hossain et al., 1995. Identification of gene products encoded by the latency-related gene of bovine herpesvirus 1. J. Virol. 69:5345-5352.

Inman et al., 2002. A mutation in the latency related gene of bovine herpesvirus 1 interferes with the latency-reactivation cycle of latency in calves. J. Virol. 76:6771-6779.

Inman et al., 2001A. Region of herpes simplex virus type 1 latency-associated transcript sufficient for wild-type spontaneous reactivation promotes cell survival in tissue culture. J. Virol. 75:3636-3646.

Inman et al., 2001 B. A mutation in the latency-related gene of bovine herpesvirus 1 leads to impaired ocular shedding in acutely infected calves. J. Virol. 75:8507-8515.

Inman et al., 2001C. The zinc ring finger in the bICP0 protein encoded by bovine herpes virus-i mediates toxicity and activates productive infection. J. Gen. Virol. 82:483-492.

Jiang et al., 1998. A protein encoded by the latency-related gene of bovine herpesvirus 1 is expressed in trigeminal ganglionic neurons of latently infected cattle and interacts with cyclin-dependent kinase 2 during productive infection. J. Virol. 72:8133-8142.

Jones 1998. Alphaherpesvirus latency: its role in disease and survival of the virus in nature. Adv. Virus Res. 51:81-133.

Jones et al., 2000. Analysis of latency in cattle after inoculation with a temperature sensitive mutant of bovine herpesvirus 1 (RLB106). Vaccine 18:3185-3195.

Koppel et al., 1996. Recombinant bovine herpesvirus-1 (BHV-1) lacking transactivator protein BICPO entails lack of glycoprotein C and severely reduced infectivity. Biol. Chem. 377:787-795.

Kutish et al., 1990. Characterization of the latency-related transcriptionally active region of the bovine herpesvirus 1 genome. J. Virol. 64:5730-5737.

Leib et al., 2000. Specific phenotypic restoration of an attenuated virus by knockout of a host resistant gene. Proc. Natl. Acad. Sci. USA 97:6097-6101.

Maggioncalda et al., 1996. Correlation between herpes simplex virus type 1 rate of reactivation from latent infection and the number of infected neurons in trigeminal ganglia. Virology 225:72-81.

Naturaj et al., 1997. Bovine herpesvirus 1 downregulates the expression of bovine MHC class I molecules. Viral Immunol. 10:21-34.

Perng et al., 2002. A gene capable of blocking apoptosis can substitute for the herpes simplex virus type 1 latency-associated transcript gene and restore wild-type reactivation levels. J. Virol. 76:1224-1225.

Perng et al., 2000A. Virus-induced neuronal apoptosis blocked by the herpes simplex virus latency-associated transcript (LAT). Science 287:1500-1503.

Perng et al., 2000B. The latency-associated transcript gene enhances establishment of herpes simplex virus type 1 latency in rabbits. J. Virol. 74:1885-1891.

Perng et al., 1994. The latency-associated transcript gene of herpes simplex virus type 1 (HSV-1) is required for efficient in vivo spontaneous reactivation of HSV-1 from latency. J. Virol. 68:8045-8055.

Perng et al., 1996. A 371-nucleotide region between the herpes simplex virus type 1 (HSV-1) LAT promoter and the 2-kilobase LAT is not essential for efficient spontaneous reactivation of latent HSV-1. J. Virol. 70:2014-2018.

Perng et al., 1999. Herpes simplex virus type 1 serum neutralizing antibody titers increase during latency in rabbits latently infected with latency-associated transcript (LAT)-positive but not LAT-negative viruses. J. Virol. 73:9669-9672.

Perng et al., 1995. An avirulent ICP34.5 deletion mutant of herpes simplex virus type 1 is capable of in vivo spontaneous reactivation. J. Virol. 69:3033-3041.

Rock et al., 1992. Characterization of dexamethasone-induced reactivation of latent bovine herpesvirus 1. J. Virol. 66:2484-2490.

Rock et al., 1987A. Mapping bovine herpesvirus type 1 latency-related RNA in trigeminal ganglia of latently infected rabbits. J. Virol. 61:3827-3831.

Rock et al., 1987B. Detection of latency-related viral RNAs in trigeminal ganglia of rabbits latently infected with herpes simplex virus type 1. J. Virol. 61:3820-3826.

Sawtell, 1997. Comprehensive quantification of herpes simplex virus latency at the single-cell level. J. Virol. 71:5423-5431.

Sawtell and Thompson, 1992. Herpes simplex virus type 1 latency-associated transcription unit promotes anatomical site-dependent establishment and reactivation from latency. J. Virol. 66:2157-2169.

Schang and Jones, 1997. Analysis of bovine herpesvirus 1 transcripts during a primary infection of trigeminal ganglia of cattle. J. Virol. 71:6786-6795.

Schang et al., 1996. The latency-related gene of bovine herpesvirus 1 encodes a product which inhibits cell cycle progression. J. Virol. 70:3807-3814.

Schwyzer and Ackermann, 1996. Molecular virology of ruminant herpesviruses. Vet. Microbiol. 53:17-29.

Sheffy and Davies, 1972. Reactivation of a bovine herpesvirus after corticosteroid treatment. Proc. Soc. Exp. Biol. Med. 140:974-976.

Stevens et al., 1987. RNA complementary to a herpesvirus alpha gene mRNA is prominent in latently infected neurons. Science 235:1056-1059.

Thomas et al., 1999. Herpes simplex virus latency-associated transcript encodes a protein which greatly enhances virus growth, can compensate for deficiencies in immediate-early gene expression, and is likely to function during reactivation from virus latency. J. Virol. 73:6618-6625.

Thompson and Sawtell, 1997. The herpes simplex virus type 1 latency-associated transcript gene regulates the establishment of latency. J. Virol. 71:5432-5440.

Tikoo et al., 1995. Bovine herpesvirus 1 (BHV-1): biology, pathogenesis, and control. Adv. Virus Res. 45:191-223.

Wagner and Bloom, 1997. Experimental investigation of herpes simplex virus latency. Clin. Microbiol. Rev. 10:419-443.

Winkler et al., 1999. Bovine herpesvirus 1 can infect CD4⁺ T lymphocytes and induce programmed cell death during acute infection of cattle. J. Virol. 73:8657-8668.

Winkler et al., 2000. Persistence and reactivation of bovine herpesvirus 1 in the tonsil of latently infected calves. J. Virol. 74:5337-5346.

Wirth et al., 1992. Immediate-early RNA 2.9 and early RNA 2.6 of bovine herpesvirus 1 are 3′ coterminal and encode a putative zinc finger transactivator protein. J. Virol. 66:2763-2772.

Wirth et al., 1991. The three major immediate-early transcripts of bovine herpesvirus 1 arise from two divergent and spliced transcription units. J. Virol. 65:195-205.

Wirth et al., 1989. Spatial and temporal distribution of bovine herpesvirus 1 transcripts. J. Virol. 63:4882-4889.

Wyler et al., 1989. Infections bovine rhinotracheitis/vulvovaginitis (BHV-1), p. 1-72. In G. Witman (ed.), Herpesvirus diseases of cattle, horses, and pigs. Kluwer Academic Publishers, Boston, Mass.

Zwaagstra et al., 1990. Activity of herpes simplex virus type 1 latency-associated transcript (LAT) promoter in neuron-derived cells: Evidence for neuron specificity and for a large LAT transcript. J. Virol. 64:5019-5028.

What is claimed and desired to be secured by Letters Patent is as follows:

1. A vaccine composition against a viral or bacterial pathogen, comprising a viral vector, wherein said viral vector is a mutant recombinant BHV-1 strain comprising:
a. a mutation in the latency-related (LR) gene, whereby no detectable reactivation from latency occurs; and
b. an expressable recombinant nucleic acid encoding an antigen from said viral or bacterial pathogen, wherein expression of said antigen induces immunological response against said viral or bacterial pathogen.

2. The vaccine composition of claim 1, wherein said viral vector comprises modified BHV-1 strain on deposit with the American Type Culture Collection as Accession No. ______.

3. The vaccine composition of claim 1, wherein said mutation in the LR gene is accomplished by chemical synthesis or artificial manipulation of isolated segments of nucleic acid.

4. The vaccine composition of claim 1, wherein said mutation in the LR gene is spontaneous or a selected, naturally occurring mutation.

5. The vaccine composition of claim 1, wherein the live, attenuated BHV-1 strain comprises a modified BHV-1 strain other than the BHV-1 strain on deposit with the American Type Culture Collection as Accession No. VR-864.

6. The vaccine composition of claim 1, wherein said mutated LR gene comprises the nucleotide sequence of SEQ ID NO:8.

7. The vaccine of claim 1, wherein said expressable recombinant nucleic acid is inserted in said mutated LR gene locus, whereby expression of the recombinant nucleic acid is driven by the LR promoter.

8. The vaccine of claim 1, wherein said expressable recombinant nucleic acid is inserted in a glycoprotein locus of the viral vector, whereby expression of the recombinant nucleic acid is driven by a heterologous promoter.

9. The vaccine composition of claim 1, wherein said vaccine is adapted for administration by a mode selected from the group consisting of intranasal instillation, intramuscular, ocular, intraperitoneal, and subcutaneous.

10. The vaccine composition of claim 1, wherein said viral or bacterial pathogen is selected from the group consisting of bovine herpesvirus 1, bovine viral diarrhea virus, bovine respiratory virus, bovine corona virus, Pasteurella haemolytica, Pasteurella multocida, and Haemophilus somnus.

11. A method of modulating the immune response to a viral or bacterial infection, which comprises administering a vaccine composition according to claim 1.

12. A method of vaccinating a mammal in need thereof against a viral or bacterial pathogen, which comprises administering to said mammal a vaccine composition according to claim 1.

13. The method of claim 12, wherein said mammal is an ungulate.

14. The method of claim 13, wherein said ungulate is selected from the group consisting of bovines, buffalo, sheep, and deer.

15. The method of claim 12, wherein said viral or bacterial pathogen is selected from the group consisting of bovine herpesvirus 1, bovine viral diarrhea virus, bovine respiratory virus, bovine corona virus, Pasteurella haemolytica, Pasteurella multocida, and Haemophilus somnus.

16. A recombinant viral vector comprising SEQ ID NO:8 and a recombinant nucleic acid fragment capable of encoding an antigen from a viral or bacterial pathogen.

17. The recombinant viral vector of claim 16, wherein said viral or bacterial pathogen is selected from the group consisting of bovine herpesvirus 1, bovine viral diarrhea virus, bovine respiratory virus, bovine corona virus, Pasteurella haemolytica, Pasteurella multocida, and Haemophilus somnus.

18. A method for effecting the treatment of a viral or bacterial infection, which comprises administering to a mammal in need of said treatment, an effective amount of the vaccine of claim 1.

19. The method of claim 18, wherein said mammal is an ungulate.

20. The method of claim 19, wherein said ungulate is selected from the group consisting of bovines, buffalo, sheep, and deer.

21. The method of claim 18, wherein said viral or bacterial pathogen is selected from the group consisting of bovine herpesvirus 1, bovine viral diarrhea virus, bovine respiratory virus, bovine corona virus, Pasteurella haemolytica, Pasteurella multocida, and Haemophilus somnus


SEQUENCE LISTING (8 sequences)

SEQ ID NO: 1 - DNA, 23 bp, Artificial
  p4 primer
cgtgtatttgcgacccccagcct

SEQ ID NO: 2 - DNA, 20 bp, Artificial
  p5 primer
gccagaccaaaccccccgca

SEQ ID NO: 3 - DNA, 18 bp, Artificial
  forward gC primer
aaagccccgccgaaggag

SEQ ID NO: 4 - DNA, 19 bp, Artificial
  reverse gC primer
tacgaacagcagcacgggc

SEQ ID NO: 5 - DNA, 19 bp, Artificial
  forward gH primer
gctttcgccctgctctgcc

SEQ ID NO: 6 - DNA, 19 bp, Artificial
  reverse gH primer
tcctgcctccccaccccta

SEQ ID NO: 7 - DNA, 37 bp, Bovine herpesvirus 1
gcatgcgcgacctgggccataaaagccccgcgcatgc

SEQ ID NO: 8 - DNA, 28 bp, Artificial
  mutant oligonucleotide
gcatgcagaattctagttagttagcatg